(19) United States
(12) Patent Application Publication
(10) Pub. No.: US 2016/0302810 A1
(43) Pub. Date: Oct. 20, 2016

(54) Surgical Tool

(71) Applicant: Whitney Surgical Labs, LLC, Bellingham, WA (US)

(72) Inventor: Timothy M. Whitney, Bellingham, WA (US)

(73) Assignee: Whitney Surgical Labs, LLC, Bellingham, WA (US)

(21) Appl. No.: 15/085,930

(22) Filed: Mar. 30, 2016

Related U.S. Application Data
(60) Provisional application No. 62/148,377, filed on Apr. 16, 2015.

(51) Int. Cl. A61B 17/29; A61B 17/00

(52) U.S. Cl. CPC A61B 17/29; A61B 17/00234; A61B 2017/00292; A61B 2017/00367; A61B 2017/00424; A61B 2017/2903; A61B 2017/2905; A61B 2017/2923; A61B 2017/2932

(57) ABSTRACT

A surgical tool has a base portion, a tool portion, and a transfer portion. The base portion defines a base axis and comprises at least one trigger member. The tool portion defines a tool axis and comprises at least one tool assembly. The transfer portion is operatively connected between the base portion and the tool portion such that a pivot angle between the base axis and the tool axis may be altered and movement of the at least one trigger member causes movement of the tool assembly.

### RELATED APPLICATION

This application (Attorney's Ref. No. P218775) claims benefit of U.S. Provisional Application Ser. No. 62/148,377 filed Apr. 16, 2015, the contents of which are incorporated herein by reference.

### TECHNICAL FIELD

The present invention relates to surgical tools and, more specifically, to tools adapted for use during laparoscopic and arthroscopic surgical procedures.

### BACKGROUND

Surgical procedures can require the surgeon to perform very precise movements for extended periods of time. The need exists for ergonomic surgical tools that reduce strain on the surgeon during surgery.

### SUMMARY

The present invention may be embodied as a surgical tool comprising a base portion, a tool portion, and a transfer portion. The base defines a base axis and comprises at least one trigger member. The tool portion defines a tool axis and comprises at least one tool assembly. The transfer portion is operatively connected between the base portion and the tool portion such that a pivot angle between the base axis and the tool axis may be altered and movement of the at least one trigger member causes movement of the tool assembly.

The present invention may also be embodied as a surgical tool for use by a surgeon having a hand defining a first secondary axis defined by movement of a surgeon's fingers and a second secondary axis defined by movement of a surgeon's thumb. The surgical tool comprises a based portion, a tool portion, and a transfer portion. The base portion defines a base axis and comprises first and second trigger members. The tool portion defines a tool axis and comprises at least one tool assembly. The transfer portion is operatively connected between the base portion and the tool portion such that movement of the at least one trigger member causes movement of the tool assembly. Further, during use of the surgical tool, the first trigger member is supported for movement along the first secondary axis, and the second trigger member is supported for movement along the second secondary axis.

The present invention may also be embodied as a method of allowing a surgeon to perform surgery. The surgeon has a hand defining a first secondary axis defined by movement of a surgeon's fingers and a second secondary axis defined by movement of a surgeon's thumb to perform surgery. The method comprises the following steps. A base portion defining a base axis is provided. First and second trigger members are supported for movement relative to the base portion. A tool portion defining a tool axis and comprising at least one tool assembly is provided. The base portion and the tool portion are operatively connected such that movement of the first and second trigger members causes movement of the tool assembly. The base portion is held in the surgeon's hand such that the first trigger member is supported for movement along the first secondary axis and the second trigger member is supported for movement along the second secondary axis. The first and second trigger members are displaced along the first and second secondary axes to operate the at least one tool assembly.

### BRIEF DESCRIPTION OF THE DRAWINGS

FIG. 1 is a side elevation view of a first example surgical tool of the present invention being held by a surgeon's hand;

FIG. 2 is top plan partial view of the first example surgical tool;

FIG. 3 is a side elevation partial view of the first example surgical tool;

FIG. 4A is a front elevation view of the surgeon's hand illustrating a primary axis, a first secondary hand axis, and a second secondary hand axis defined by the surgeon's hand;

FIG. 4B is a front elevation view of the surgeon's hand illustrating movement of the surgeon's fingers and thumb along the first secondary hand axis towards the primary axis and the surgeon's thumb along the second secondary hand axis towards the primary axis;

FIG. 5 is a top plan view of a surgeon illustrating a position of the surgeon's hand relative to the surgeon's forearm when the hand is in a neutral, comfortable position;

FIG. 6 is a front elevation view illustrating how the first example surgical tool is arranged relative to the primary axis and the first and second secondary axes defined by the surgeon's hand;

FIGS. 7 and 8 are top plan, partial cutaway views depicting a base portion of the first example tool in first and second base configurations, respectively;

FIGS. 9 and 10 are front elevation cutaway views taken along lines 9-9 in FIG. 7 depicting the base portion of the first example tool in the first and second base configurations, respectively;

FIGS. 11 and 12 are top plan, partial cutaway views depicting a transfer portion of the first example tool in first and second transfer configurations, respectively;

FIGS. 13 and 14 are side elevation, partial cutaway views depicting the transfer portion of the first example tool in the first and second transfer configurations, respectively;

FIG. 15 is a front elevation cutaway view taken along lines 15-15 in FIG. 11 depicting the base portion of the first example tool in the first base configuration;

FIG. 16 is a side elevation view depicting the transfer portion of the first example tool in a coupled configuration;

FIG. 17 is a side elevation view depicting the transfer portion of the first example tool in an uncoupled configuration;

FIG. 18 is a side elevation view depicting the transfer portion of the first example tool being rotated relative to the base portion when in the uncoupled configuration;

FIG. 19 is a side elevation, partial cutaway view depicting the transfer portion of the first example tool in the uncoupled configuration;

FIG. 20 is a side elevation view depicting the transfer portion of the first example tool being moved between first and second limits when in the uncoupled configuration;

FIG. 21 is a side elevation view depicting a portion of a second example tool of the present invention;

FIG. 22 is a top plan view depicting a portion the second example tool;

FIG. 23 is top plan partial view of a third example surgical tool of the present invention;

FIG. 24 is a side elevation partial view of a fourth example surgical tool;

FIG. 25 is a top plan view of the fourth example tool in a first base configuration;

FIG. 26 is a top plan, partial cutaway view depicting a base portion of the fourth example tool in a first base configuration;

FIGS. 27 and 28 are front elevation cutaway views taken along lines 27-27 and 28-28, respectively, in FIG. 26 depicting the base portion of the fourth example tool in the first base configuration;

FIG. 29 is a top plan, partial cutaway view depicting a base portion of the fourth example tool in a second base configuration;

FIGS. 30 and 31 are front elevation cutaway views taken along lines 30-30 and 31-31, respectively, in FIG. 29 depicting the base portion of the fourth example tool in the second base configuration;

FIG. 32 is a top plan, partial cutaway view depicting a base portion of a fifth example tool in a first base configuration;

FIG. 33 is a front elevation cutaway view taken along line 33-33 in FIG. 32 depicting the base portion of the fifth example tool in a first base configuration;

FIG. 34 is a view similar to FIG. 33 of the fifth example tool in a second base configuration;

FIG. 35 is a side elevation view illustrating the use of a sixth example tool;

FIG. 36 is a front section view of the sixth example tool as used;

FIG. 37 is a side elevation view depicting the transfer portion of the seventh example tool being moved between first and second limits; and

FIG. 38 is a side elevation view depicting the transfer portion of the eighth example tool being moved between first and second limits.

### DETAILED DESCRIPTION

The present invention is a surgical tool that may be embodied in a number of different forms, and several example surgical tools constructed in accordance with, and embodying, the principles of the present invention will be described below.

### I. First Example Surgical Tool

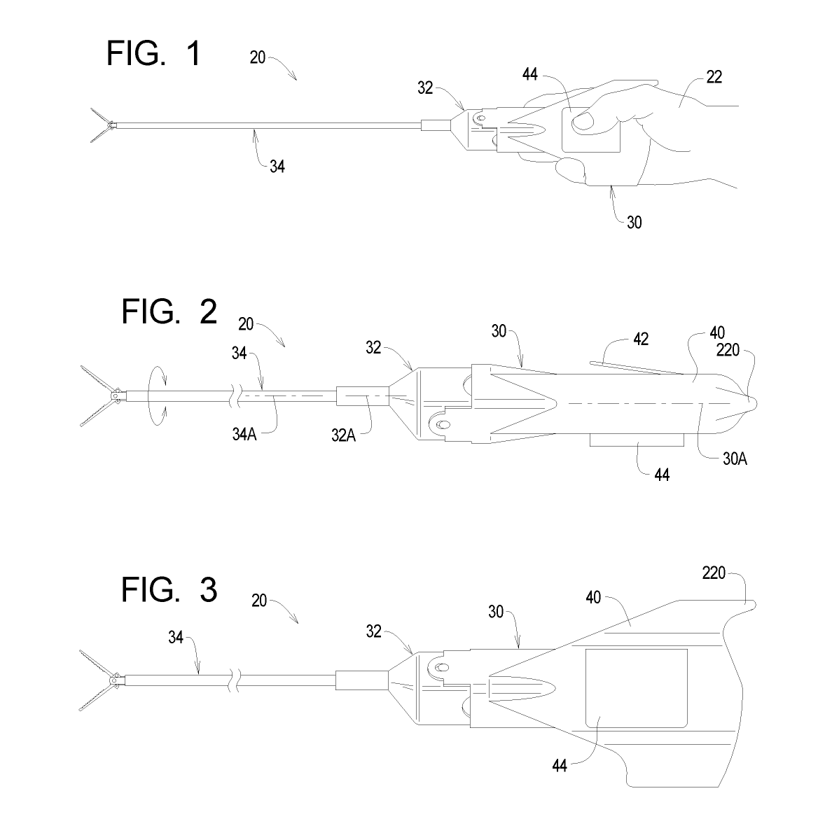

Referring initially to FIGS. 1-5 of the drawing, depicted therein is a first example surgical tool 20 constructed in accordance with, and embodying, the principles of the present invention. The example surgical tool 20 is intended to be held by a surgeon for use during laparoscopic and arthroscopic surgery, and a hand 22 of the surgeon is depicted in FIGS. 1 and 4-5.

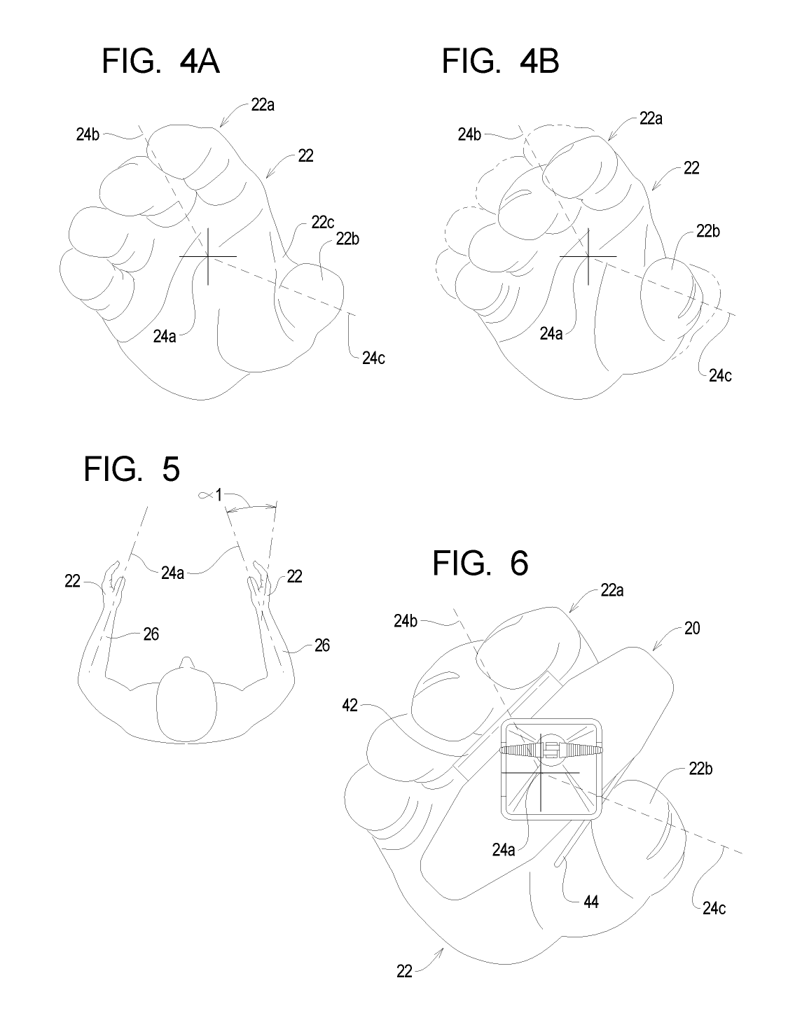

FIGS. 4A and 4B illustrate that the surgeon's hand defines a primary axis 24A and first and second secondary axes 24b and 24c. As perhaps best shown in FIG. 5, when the surgeon's hand 22 is in such a neutral, comfortable position, the primary axis 24A is at a slight angle α1 relative to the surgeon's forearm 26. FIG. 5 also shows that, when the surgeon's hand 22 is in a neutral, comfortable position, the elbows (not visible) are held in and the forearms 26 are angled slightly inward (similar to the position of the hand during a handshake). Although not explicitly shown in FIGS. 4A, 4B, and 5, the surgeon's shoulders will typically be neutral to slightly externally rotated and back and down when the surgeon's hand 22 is in the neutral, comfortable position.

Laparoscopic and arthroscopic surgery may require the surgeon to operate for many minutes or even hours without break. If the surgeon's hand is not in a neutral, comfortable position during surgery, the surgeon may suffer reduced stamina in the short term and develop repetitive use injuries in the long term. One purpose of the first example surgical tool 20 is to allow the surgeon's hand 22 to be in such a neutral, comfortable when the tool 20 is being used to avoid such repetitive use injuries. Another purpose of the first example surgical tool 20 is to allow the tool 20 to be reconfigured as the surgical procedure progresses.

Referring for a moment back to FIGS. 1-3 of the drawing, it can be seen that the first example surgical tool 20 comprises a base portion 30 defining a base axis 30A, a transfer portion 32 defining a transfer axis 32A, and a tool portion 34 defining a tool axis 34A. With the base portion 30 grasped by the surgeon's hand 22, a neutral pinching motion of the user's fingers 22a towards the user's thumb 22b causes the fingers 22a to move along the first secondary hand axis 26 and the thumb 22b to move along the second secondary hand axis 28.

And as will be described in further detail below, the transfer axis 32A and thus the tool axis 34A may be placed at a pivot angle relative to the base axis 30A such that the surgeon may hold the first example surgical tool 20 in the neutral, comfortable position during most surgical situations. In particular, during use the tool axis 34A is fixed relative to the transfer axis 32A such that the angle at which the tool axis 34A extends relative to the base axis 30A may also be changed. Further, the tool portion 34 may be axially rotated about the tool axis 34A. The first example surgical tool 20 thus may be reconfigured as necessary to reduce stress on the surgeon's hand 22 and thus the likelihood of repetitive use injuries to the surgeon.

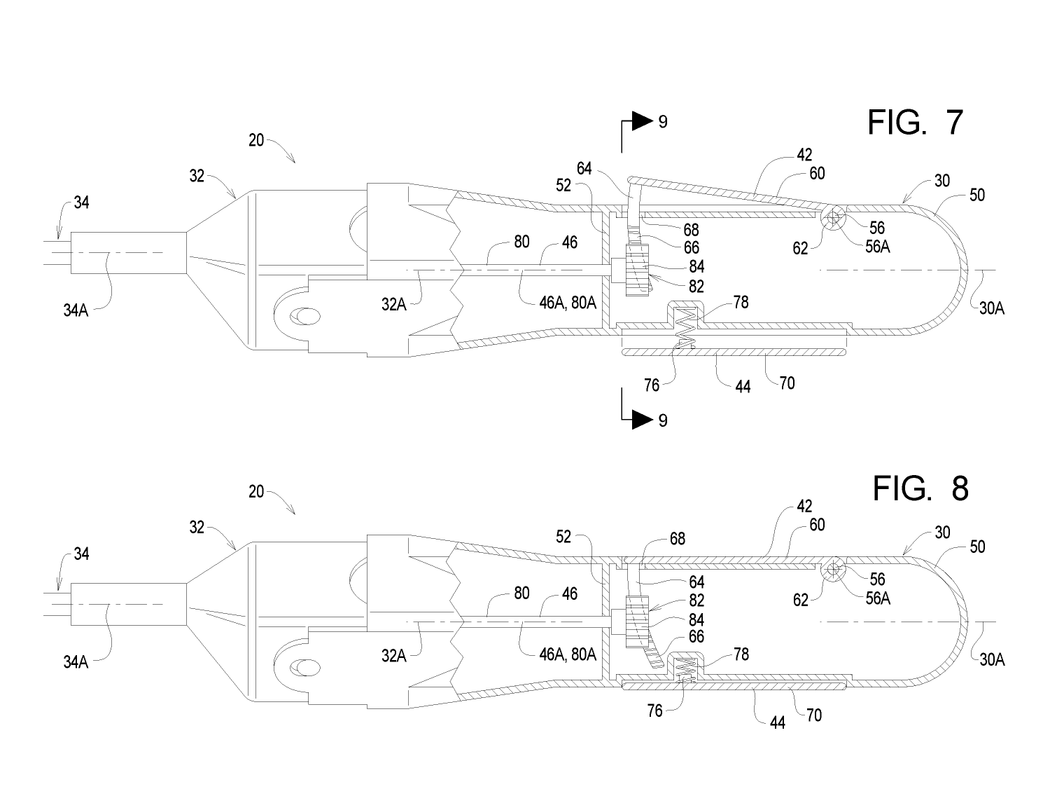

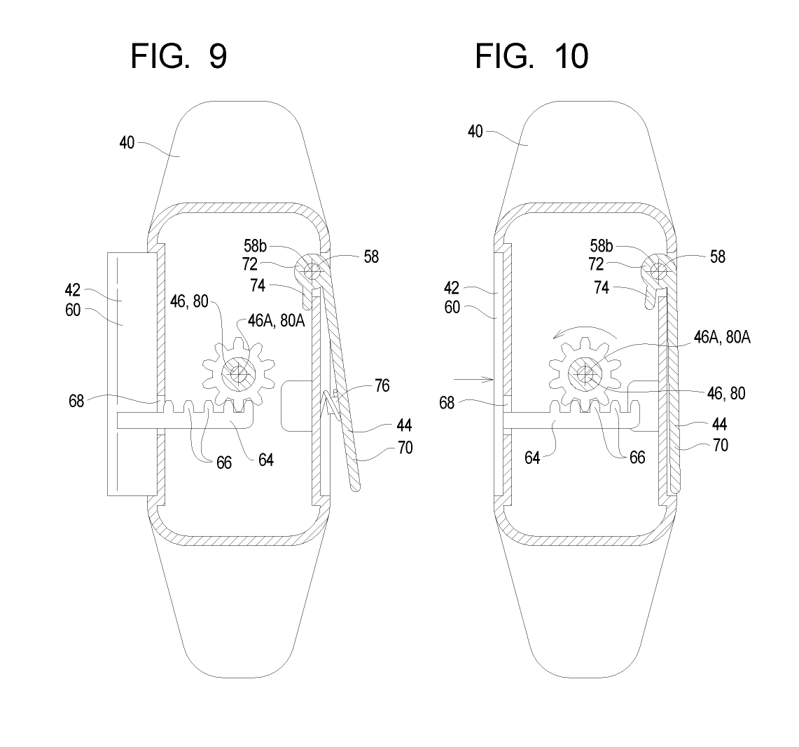

Referring now to FIGS. 7-9, the example base portion 30 will now be described in further detail. The example base portion 30 comprises a base housing 40, a first trigger member 42, a second trigger member 44, and a drive shaft assembly 46 defining a drive shaft axis 46A. The base housing 40 comprises a base housing outer wall 50 and first and second interior walls 52 and 54. The base outer wall 50 supports first and second pivot pins 56 and 58 defining a first trigger axis 56A and a second trigger axis 58A, respectively. The example first and second trigger axes 56A and 56B are substantially perpendicular to each other as shown, for example, by a comparison of FIGS. 7 and 8 with FIGS. 9 and 10, respectively.

The first trigger member 42 defines a first trigger plate 60, a first pivot structure 62, and a rack projection 64 defining a plurality of rack teeth 66. A rack opening 68 is formed in the base outer wall 50. The second trigger member 44 defines a second trigger plate 70, a second pivot structure 72, a catch extension 74, and a spring nub 76.

The first and second pivot structures 62 and 72 receive the first and second pivot pins 56 and 58 such that the first and second trigger plates 60 and 70 are pivotably supported to the base housing outer wall 50. So supported, the rack projection 64 extends through the rack opening 68 and interior of the base housing 40. A trigger spring 78 is supported by the spring nub 76 between the base housing 40 and the second trigger plate 70 to resiliently oppose movement of the second trigger plate 70 towards the base housing 40.

The drive shaft assembly 46 comprises a drive shaft 80 defining a drive axis 80A, a drive gear 82 defining radial drive teeth 84, and a first transfer gear 86 defining a first set of conical teeth 88. The drive shaft assembly 46 is supported by the first and second interior walls 52 and 54 for axial rotation about the drive shaft axis 46A.

With the drive shaft assembly 46 so supported by the interior walls 52 and 54 and the first trigger member 42 supported by the first pivot pin 56 as described above, the rack projection 64 extends through the rack opening 68 in the base housing 40 such that the rack teeth 66 engage the radial drive teeth 84 on the drive gear 82. Further, the example rack projection 64 is arcuate or curved and the rack teeth 66 are angled relative to the rack projection 64 such that, as the first trigger plate 60 is pivoted relative to the base housing 40, the rack teeth 66 engage the radial drive teeth 84 to cause axial rotation of the drive shaft 80 about the drive shaft axis 46A. Axial rotation of the drive shaft assembly 46 results in axial rotation of the first transfer gear 86 about the drive shaft axis 46A.

With the foregoing understanding of the example base portion 30 in mind, the construction and operation of the example transfer portion 32 and the interoperation of the base portion 30 and transfer portion 32 will now be described.

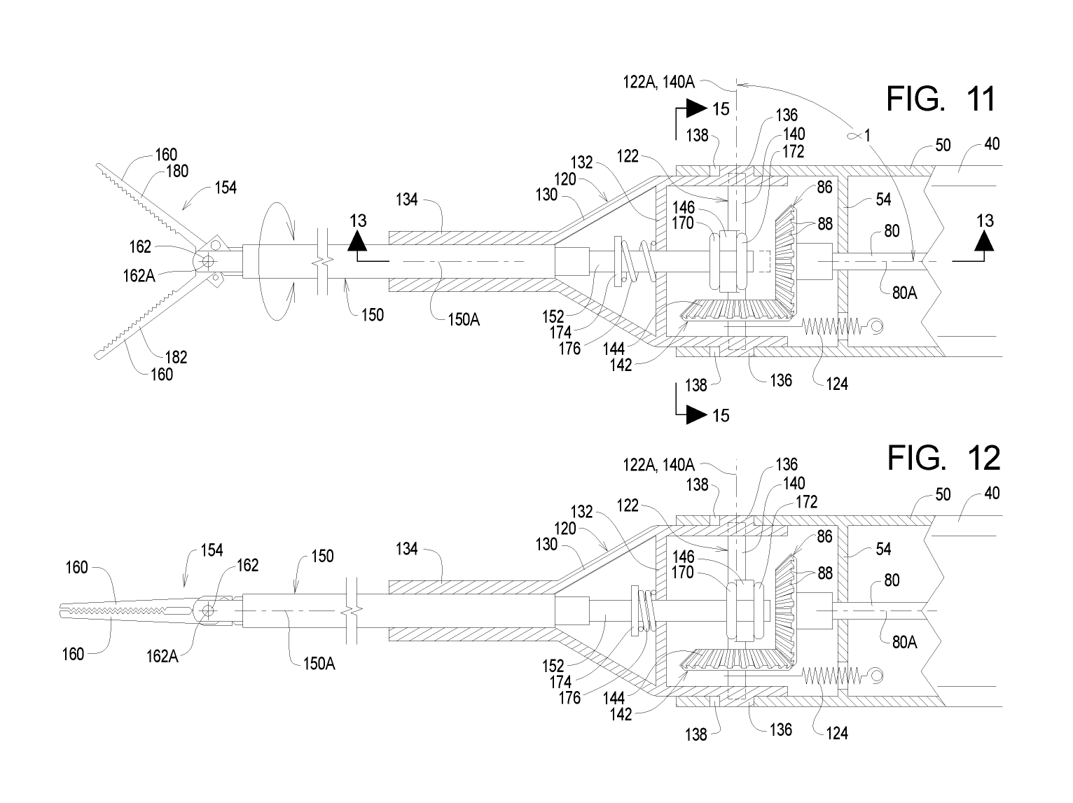

As perhaps best shown in FIGS. 11-15, the example transfer portion 32 comprises a transfer housing 120, a transfer shaft assembly 122 defining a transfer shaft axis 122A, and a transfer spring 124. The transfer housing 120 defines a transfer housing outer wall 130 and an interior support wall 132. The transfer housing outer wall 130 defines a neck portion 134 and mounting projections 136.

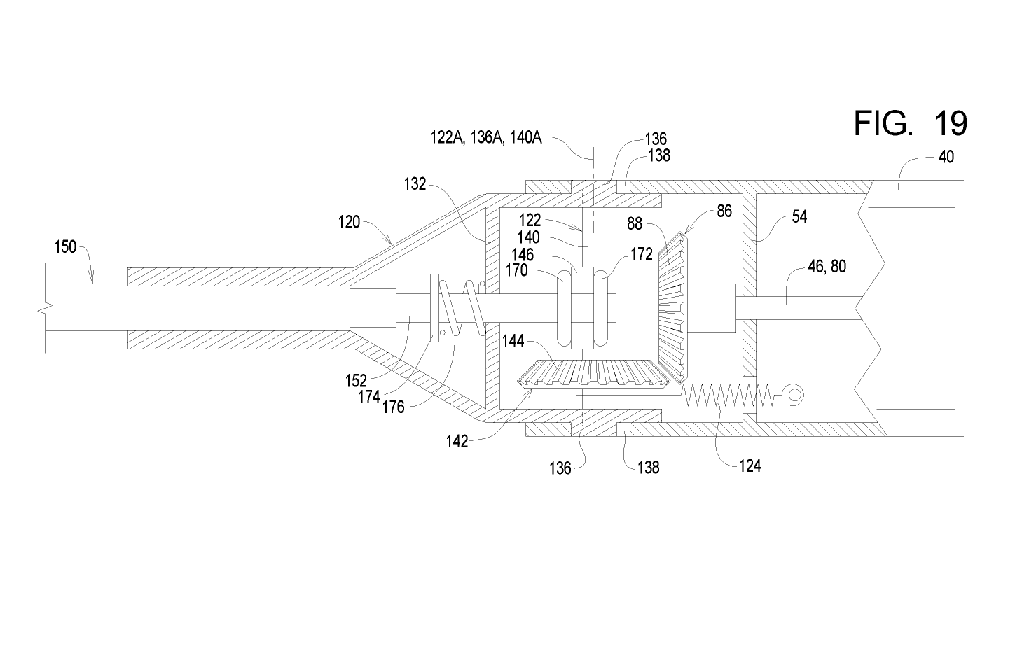

The mounting projections 136 are sized and dimensioned to be received by mounting slots 138 formed in the base housing outer wall 50. As will be described in further detail below, the mounting slots 138 allow limited movement of the transfer housing 120 relative to the base housing 40 between an engaged position (FIGS. 11-16) and a disengaged position (FIGS. 17-19). The housing slots 138 also allow pivoting of the transfer housing 120 about a housing pivot axis 136A relative to the base housing 40. The transfer spring 124 is arranged to extend between the base housing 40 and the transfer housing 120 to bias the transfer housing 120 into the engaged position. The transfer spring 124 as configured thus forms a lock assembly for maintaining the transfer housing 120 in the engaged position.

The example transfer shaft assembly 122 comprises a transfer shaft 140 defining a transfer shaft axis 140A, a second transfer gear 142 defining a second set of conical teeth 144, and at least one transfer arm extending radially from the transfer shaft axis 140A. The example transfer shaft assembly 122 comprises a transfer arm 146.

When the transfer housing 120 is in the engaged position relative to the base housing 40, the first and second transfer gears 86 and 142 are in contact such that the first set of conical teeth 88 engages the second set of conical teeth 144. The first set of conical teeth 88 extend at an angle to the drive shaft axis 80A and the second set of conical teeth 144 extend at an angle to the transfer shaft axis 140A. Accordingly, the engagement of the first and second transfer gears 86 and 142 convert axial rotation of the drive shaft 80 about the drive shaft axis 80A into axial rotation of the transfer shaft 140 about the transfer shaft axis 140A, and the transfer shaft axis 140A extends at an angle α2 relative to the drive shaft axis 80A. In the example surgical tool 20, the angle α2 is substantially 90°. Further, the transfer arm 146 pivots about the transfer shaft axis 140A with axial rotation of the transfer shaft 140 as will be described in further detail below.

With the foregoing understanding of the example base portion 30 and the example transfer portion 32 in mind, the construction and operation of the example tool portion 34 and the interoperation of the base portion 30, the transfer portion 32, and the tool portion 34 will now be described.

Referring again to FIGS. 11-15, it can be seen that the example tool portion 34 comprises a tool housing 150 defining a tool axis 150A, a tool rod or cable 152, and a tool assembly 154. The example tool housing 150 is an elongate rigid or semi-rigid member defining a cylindrical tool rod chamber and having one or more tool flanges 160 mounted on a distal end thereof. The example tool flanges 160 support a tool pin 162 defining a tool working axis 162A. The tool housing 150 is rigidly or detachably received by the neck portion 134 of the transfer housing 120 so that movement of the transfer housing 120 causes movement of the tool housing 150.

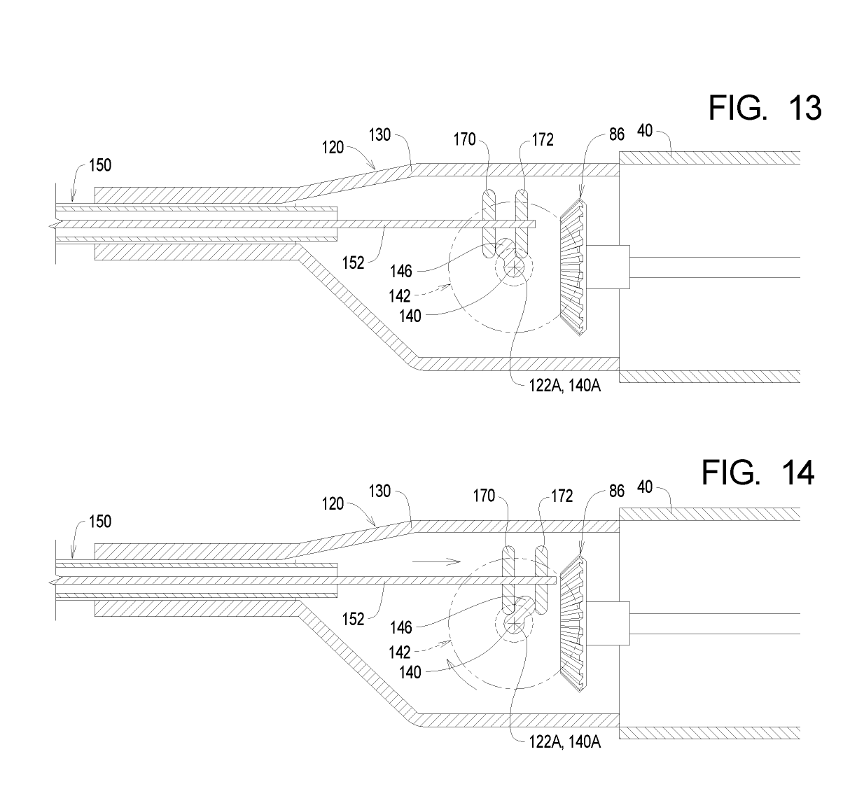

The tool rod or cable 152 is supported for movement along the tool axis 150A within the cylindrical tool rod chamber defined by the tool housing 150. As shown in FIGS. 11-14, the tool rod or cable 152 is movable between extended (FIGS. 11 and 13) and retracted (FIGS. 12 and 14) positions relative to the tool housing 150 and the transfer housing 120. First and second rod collars 170 and 172 are secured to the tool rod or cable 152 adjacent to a proximal end thereof. Also secured to the tool rod 152 is a spring flange 174. A return spring 176 is arranged between the transfer housing 120 and the spring flange 174 to bias the tool rod into the extended position.

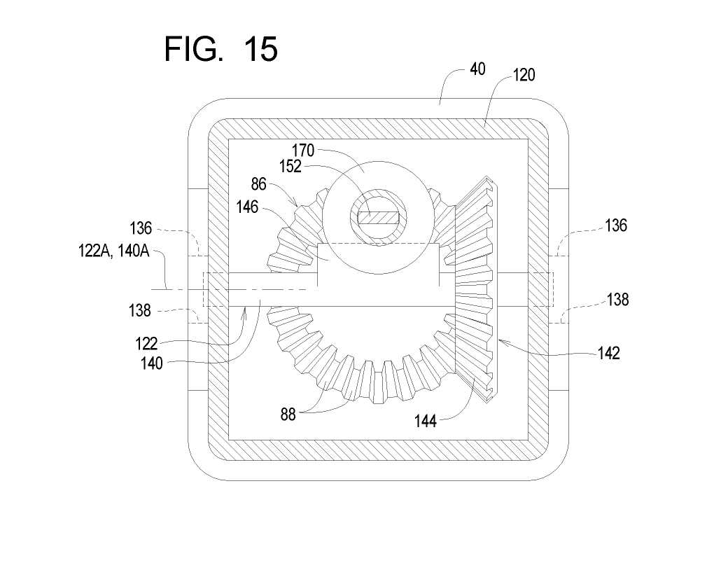

As perhaps best shown in FIG. 15 (and by comparison of FIGS. 11 and 12 with FIGS. 13 and 14), the example tool rod or cable 152 is a flat bar made of steel that is sufficiently rigid to allow a distal end thereof to be moved along the tool axis 150A with movement of the proximal end along the tool axis 150A and such that axial rotation of the distal end about the tool axis 150A cause similar axial rotation of the proximal end of the tool rod or cable 152.

In the example tool portion 34, the tool pin 162 supports first and second tool members 180 and 182 for pivoting movement relative to the tool working axis 162A. The tool members 180 and 182 are connected or linked to the tool rod or cable 152 such that displacement of the tool rod or cable 152 along the tool axis 150A moves the tool members 180 and 182 between an open configuration as shown in FIG. 11 and a closed position as shown in FIG. 12. Similarly, axial rotation of the tool housing 150 is transmitted to the tool rod or cable 152 and the tool members 180 and 182 linked thereto.

The link or connection of the tool members 180 and 182 is well-known in the art and will not be described in detail herein. The example tool members 180 and 182 are gripping members, but clamping members, cutting members, or the like may be used instead. Further, although both tool members 180 and 182 are moved by displacement of the tool rod or cable 152 in the example tool portion 34, only one of these need be movable to implement a different tool function.

FIGS. 11-15 further illustrate that, when the tool portion 34 is attached to the transfer portion 32, the tool rod or cable 152 lies between the transfer arm 146 and the transfer arm 146 extends between first and second the rod collars 170 and 172. Accordingly, pivoting of the transfer arm 146 about the transfer shaft axis 140A causes the transfer arm 146 to displace the rod collars 170 and 172 and thus displace the tool rod or cable 152 along the tool axis 150A as shown by a comparison of FIG. 11 with FIG. 12 and of FIG. 13 with FIG. 14. Additionally, the interaction of the transfer arm 146 with the rod collars 170 and 172 allows axial rotation of the tool rod or cable 152 relative to the transfer arm 146.

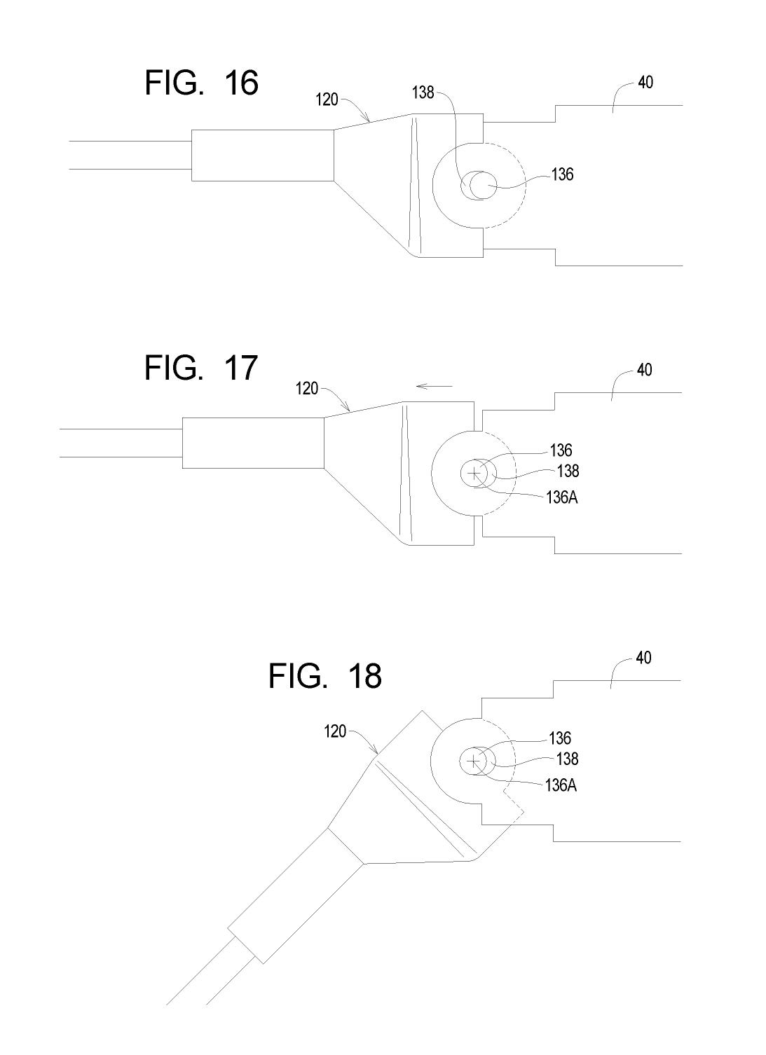

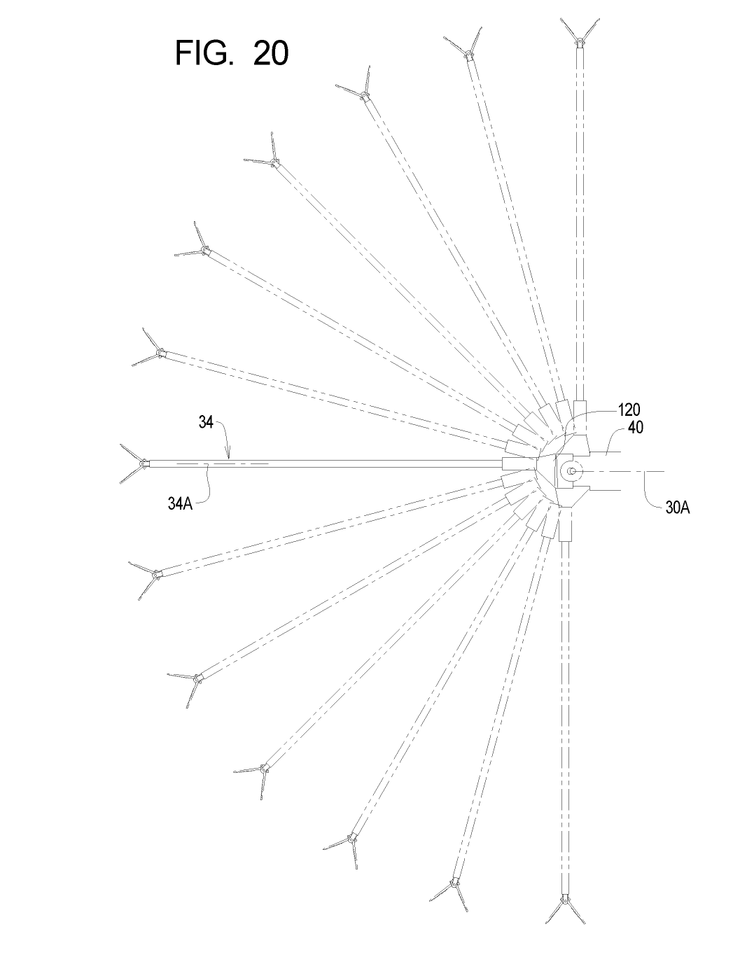

Referring now to FIGS. 16-20, it can be seen that, by displacing the transfer housing 120 into the disengaged position relative to the base housing 40 against the force of the transfer spring 124, the first and second transfer gears 86 and 142 are disengaged, allowing the transfer housing 120 to be rotated about the housing pivot axis 136A without damaging the transfer gears 86 and 142. When the transfer portion axis 32A is in a desired angular relationship with the base portion axis 30A, the transfer housing 120 is released, and the transfer spring 124 displaces the transfer housing 120 towards the base housing 40 until the second transfer gear 142 engages the first transfer gear 86. FIG. 20 illustrates that example surgical tool 20 allows the tool axis 34A to be moved within approximately a 90° arc relative to the base axis 30A. The spacing of the sets 88 and 144 of conical teeth on the transfer gears 86 and 142 determine the number of locations at which the tool axis 34A may be fixed within that 90° arc.

To use the first example surgical tool 20 as described above, the surgeon grasps the base housing 40 with the fingers 22a against the first trigger member 42 and the thumb 22b against the second trigger member 44. The surgeon then adjusts a pivot angle between the base axis 30A and the tool axis 34A as comfortable for that particular surgeon and surgical considerations. The surgeon then rotates the tool assembly 154 as determined by the needs of that particular surgery. Ideally, the surgical tool 20 will be configured so that the surgeon's hand or hands 22 can remain in a neutral, comfortable position during the performance of the surgery.

Then, the surgeon simply displaces the fingers 22a and thumb 22b along the first and second hand secondary axes 24b and 24c towards the primary axis 24a in a neutral, balance pinching motion to depress the first and second trigger members 42 and 44. The first trigger member 42 moves to rotate the drive shaft assembly 46, which in turn rotates the transfer shaft assembly 122, which in turn displaces the tool rod or cable 152 along the tool rod axis 150A to operate the tool assembly 145. The example second trigger member 44 simply allows the hand 22 to remain centered on the primary axis 24a when the first trigger plate 42 is displaced by the fingers 22a. As another option, the second trigger member 44 may be connected to a rack with rack teeth similar to the rack projection 64 and rack teeth 66 described above, in which case the movement of the second trigger member 44 may also be used to rotate the base shaft assembly 46. In any event, the second trigger member 44 is optional, and the thumb 22c may simply be braced against a fixed thumb surface formed on the base housing 40.

FIGS. 2 and 3 illustrate that the example base housing 40 defines a fixed support projection 220, and FIG. 1 illustrates that the support projection engages the palm portion 22c of the surgeon's hand 22 between the fingers 22a and thumb 22b when the surgical tool 20 is grasped by the hand 22. This support projection 220 may be used to take some of the load off of the fingers 22a and thumb 22b during use of the surgical tool 20.

### II. Second Example Surgical Tool

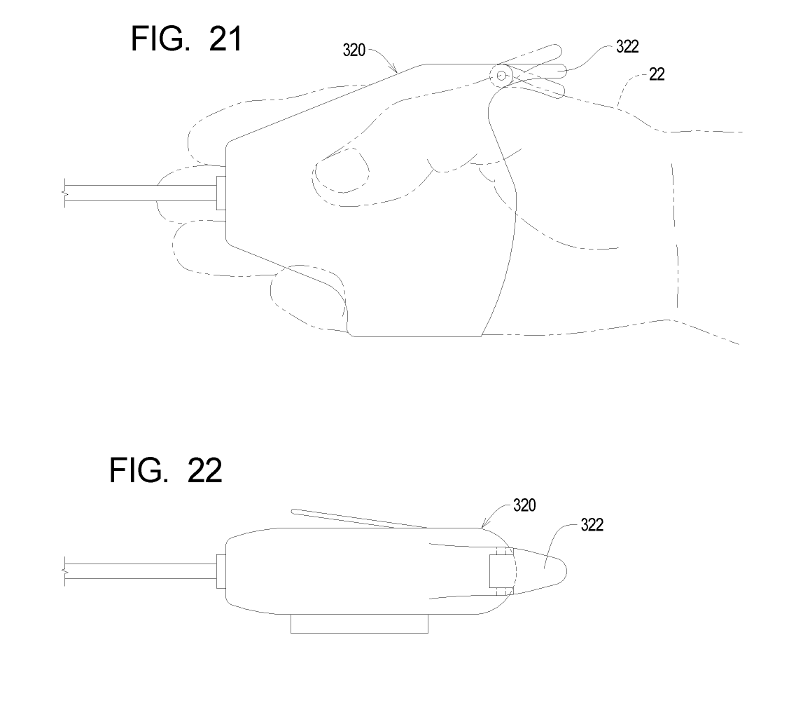

FIGS. 21 and 22 illustrate a second example surgical tool 320 similar to the first example tool 20, except that the second example surgical tool 320 employs a movable support projection 322 instead of the fixed support projection 220 described above. The movable support projection 322 is used in a manner similar to the fixed support projection 220 but can be adjusted to accommodate different hand sizes and shapes. The movable support projection 322 may also to be used as a clamp to lightly but positively clamp the second example surgical tool 320 on the hand 22, further reducing the need to support the weight of the surgical tool 320 with fingers and thumb alone.

### III. Third Example Surgical Tool

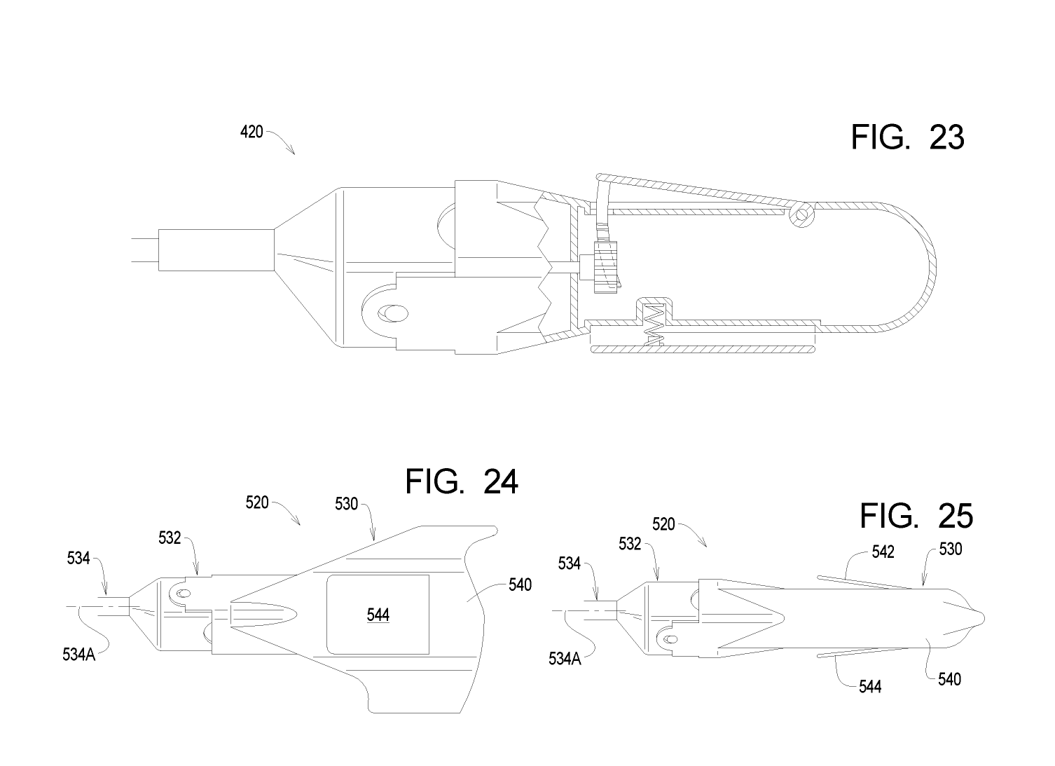

Referring now to FIG. 23 of the drawing, depicted therein is a third example surgical tool 420 constructed in accordance with, and embodying, the principles of the present invention. The fourth example surgical tool 420 is constructed and operates in the same manner as the example surgical tools 120 and 320 described above. The only difference between the tool 420 and the tools 120 described above is that the housing thereof has been shortened, providing a lighter and more compact configuration that may improve control and reduce fatigue.

### IV. Fourth Example Surgical Tool

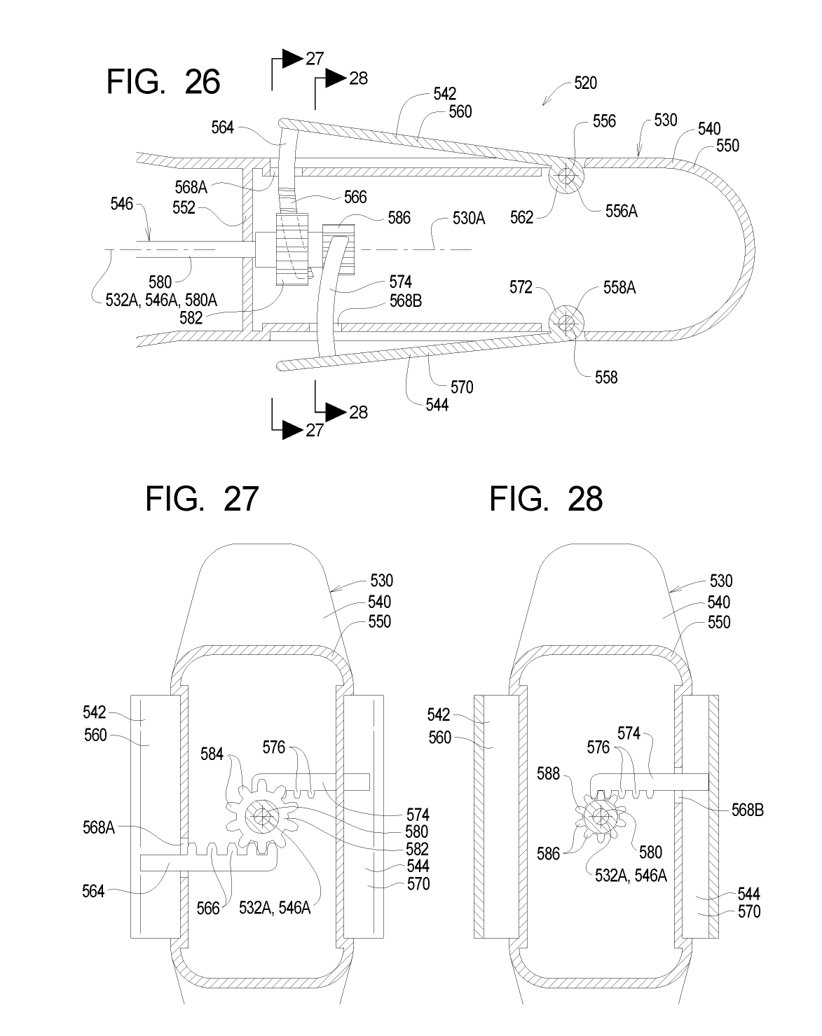

Referring now to FIGS. 24-31 of the drawing, depicted therein is a fourth example surgical tool 520 comprising a base portion 530 defining a base axis 530A, a transfer portion 532 defining a transfer axis 532A, and a tool portion 534 (FIGS. 24 and 25) defining a tool axis 534A. With the base portion 530 grasped by the surgeon's hand, a neutral pinching motion of the user's fingers towards the user's thumb causes the fingers to move along the first secondary hand axis and the thumb to move along the second secondary hand axis.

And as with the example surgical tools 120, 320, and 420, the transfer axis 532A and thus the tool axis may be placed at a pivot angle relative to the base axis 530A such that the surgeon may hold the fourth example surgical tool 520 in the neutral, comfortable position during most surgical situations. In particular, during use the tool axis is fixed relative to the transfer axis 532A such that the angle at which the tool axis extends relative to the base axis 530A may also be changed. Further, the tool portion 534 may be axially rotated about the tool axis 534A. The fourth example surgical tool 520 thus may be reconfigured as necessary to reduce stress on the surgeon's hand and thus the likelihood of repetitive use injuries to the surgeon.

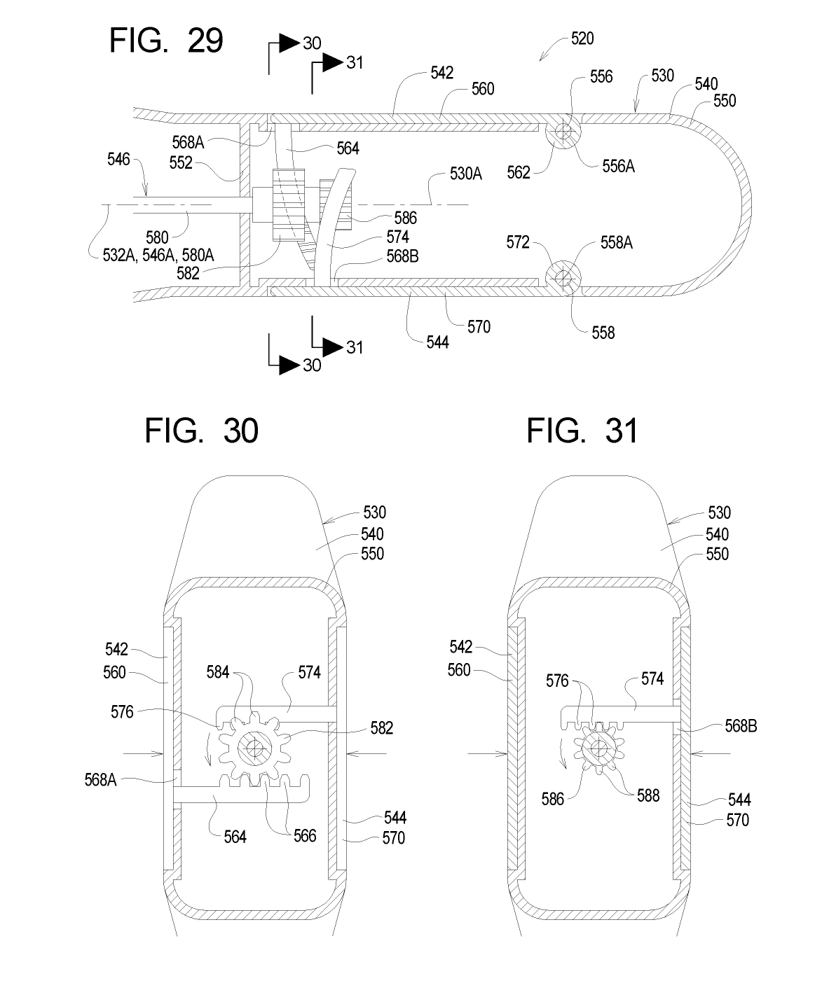

FIGS. 26-31 illustrate that the example base portion 530 comprises a base housing 540, a first trigger member 542, a second trigger member 544, and a drive shaft assembly 546 defining a drive shaft axis 546A. The base housing 540 comprises a base housing outer wall 550, a first interior wall 552 and a second interior wall (not shown). The base outer wall 550 supports first and second pivot pins 556 and 558 defining a first trigger axis 556A and a second trigger axis 558A, respectively. As perhaps best shown in FIGS. 26 and 29, the first and second trigger axes 556a and 558a are substantially perpendicular to each other.

The first trigger member 542 defines a first trigger plate 560, a first pivot structure 562, and a first rack projection 564 defining a plurality of first rack teeth 566. A first rack opening 568A is formed in one side of the base outer wall 550, while a second rack opening 568B is formed in an opposite side of the base outer wall 550. The second trigger member 544 defines a second trigger plate 570, a second pivot structure 572, and a second rack projection 574 defining a plurality of second rack teeth 576.

The first and second pivot structures 562 and 572 receive the first and second pivot pins 556 and 558 such that the first and second trigger plates 560 and 570 are pivotably supported to the base housing outer wall 550. So supported, the first and second rack projections 564 and 574 extend through the first and second rack openings 568A and 568B, respectively, and into the interior of the base housing 540. Optionally, one or more trigger springs (not shown) may be configured to resiliently oppose movement of the first and second trigger plates 560 and 570 towards each other. In the fourth example surgical tool 520, the first trigger axis 556A and the second trigger axis 558A are substantially coplanar but need not be parallel to allow movement of the surgeon's fingers and thumb along the first and second secondary axes as generally described above.

The drive shaft assembly 546 comprises a drive shaft 580 defining a drive axis 580A, a first drive gear 582 defining first radial drive teeth 584, a second drive gear 586 defining second radial drive teeth 588, and a first transfer gear (not shown) defining a first set of conical teeth. The drive shaft assembly 546 is supported by the first interior wall 552 and the second interior wall (not shown) for axial rotation about the drive shaft axis 546A.

With the drive shaft assembly 546 so supported by the interior walls and the first and second trigger members 542 and 544 supported by for pivoting movement as described above, the first and second rack projections 564 and 574 extend through the first and second rack openings 568A and 568B in the base housing 540 such that the first rack teeth 566 engage the first radial drive teeth 584 on the drive gear 582 and the second rack teeth 576 engage the second radial drive teeth 588 on the second drive gear 586. Further, the example rack projections 564 and 574 are arcuate or curved and the rack teeth 566 and 576 are angled such that, as the first and second trigger plates 560 and 570 are pivoted relative to the base housing 540, the rack teeth 566 and 576 engage the radial drive teeth 584 and 588, respectively. The sizes and dimensions of the rack teeth 566 and 576 and the radial drive teeth 584 and 588 is such that displacing the trigger plates 560 and 570 towards each other results in axial rotation of the drive shaft 580 about the drive shaft axis 546A. Axial rotation of the drive shaft assembly 546 in turn results in axial rotation of the first transfer gear about the drive shaft axis 546A.

The fourth example surgical tool 520 may be used in a manner substantially the same as the first and second example surgical tools 20 and 320 as described above. The use of two sets of rack projections 564 and 574 determines the activation force on the tool portion provided the surgeon's fingers against one of the trigger members 542 and 544 and the opposing activation force by the surgeon's thumb against the other of the trigger members 542 and 544. These opposing activation forces may be balanced or may be biased towards one or the other of the fingers or the thumb.

### V. Fifth Example Surgical Tool

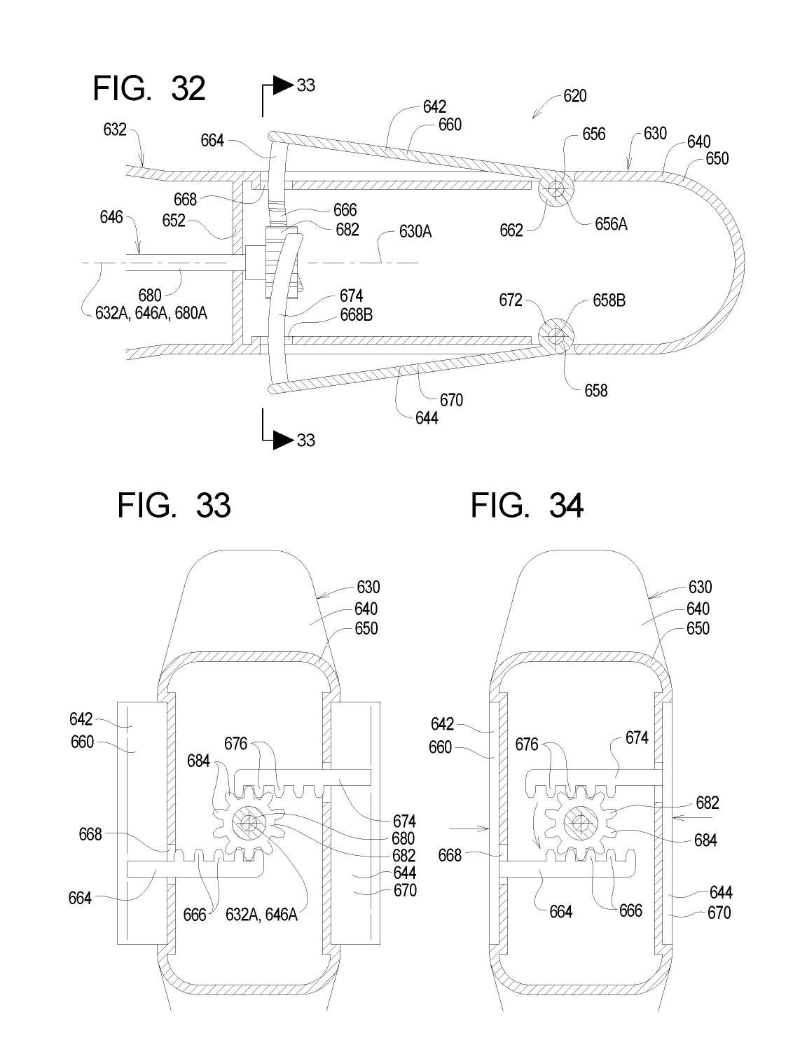

Referring now to FIGS. 32-34 of the drawing, depicted therein is a fifth example surgical tool 620 comprising a base portion 630 defining a base axis 630A, a transfer portion 632 defining a transfer axis 632A, and a tool portion (not shown) defining a tool axis. With the base portion 630 grasped by the surgeon's hand, a neutral pinching motion of the user's fingers towards the user's thumb causes the fingers to move along the first secondary hand axis and the thumb to move along the second secondary hand axis.

And as with the example surgical tools 120, 320, 420, and 520 the transfer axis 632A and thus the tool axis may be placed at a pivot angle relative to the base axis 630A such that the surgeon may hold the fifth example surgical tool 620 in the neutral, comfortable position during most surgical situations. In particular, during use the tool axis is fixed relative to the transfer axis 632A such that the angle at which the tool axis extends relative to the base axis 630A may also be changed. Further, the tool portion (not shown) may be axially rotated about the tool axis. The fifth example surgical tool 620 thus may be reconfigured as necessary to reduce stress on the surgeon's hand and thus the likelihood of repetitive use injuries to the surgeon.

FIGS. 32-34 illustrate that the example base portion 630 comprises a base housing 640, a first trigger member 642, a second trigger member 644, and a drive shaft assembly 646 defining a drive shaft axis 646A. The base housing 640 comprises a base housing outer wall 650, a first interior wall 652 and a second interior wall (not shown). The base outer wall 650 supports first and second pivot pins 656 and 658 defining a first trigger axis 656A and a second trigger axis 658A, respectively. As perhaps best shown in FIG. 32, the first and second trigger axes 656a and 658a are substantially perpendicular to each other.

The first trigger member 642 defines a first trigger plate 660, a first pivot structure 662, and a first rack projection 664 defining a plurality of first rack teeth 666. A first rack opening 668A is formed in one side of the base outer wall 650, while a second rack opening 668B is formed in an opposite side of the base outer wall 650. The second trigger member 644 defines a second trigger plate 670, a second pivot structure 672, and a second rack projection 674 defining a plurality of second rack teeth 676.

The first and second pivot structures 662 and 672 receive the first and second pivot pins 656 and 658 such that the first and second trigger plates 660 and 670 are pivotably supported to the base housing outer wall 650. So supported, the first and second rack projections 664 and 674 extend through the first and second rack openings 668A and 668B, respectively, and into the interior of the base housing 640. Optionally, one or more trigger springs (not shown) may be configured to resiliently oppose movement of the first and second trigger plates 660 and 670 towards each other. In the fifth example surgical tool 620, the first trigger axis 656A and the second trigger axis 658A are substantially coplanar but need not be parallel to allow movement of the surgeon's fingers and thumb along the first and second secondary axes as generally described above.

The drive shaft assembly 646 comprises a drive shaft 680 defining a drive axis 680A, a drive gear 682 defining radial drive teeth 684, and a first transfer gear (not shown) defining a first set of conical teeth. The drive shaft assembly 646 is supported by the first interior wall 652 and the second interior wall (not shown) for axial rotation about the drive shaft axis 646A.

With the drive shaft assembly 646 so supported by the interior walls and the first and second trigger members 642 and 644 supported for pivoting movement as described above, the first and second rack projections 664 and 674 extend through the first and second rack openings 668A and 668B in the base housing 640 such that the first and second rack teeth 666 and 676 engage the radial drive teeth 684 on the drive gear 682. Further, the example rack projections 664 and 674 are arcuate or curved and the rack teeth 666 and 676 are angled such that, as the first and second trigger plates 660 and 670 are pivoted relative to the base housing 640, the rack teeth 666 and 676 engage the radial drive teeth 684. The sizes and dimensions of the rack teeth 666 and 676 and the radial drive teeth 684 is such that displacing the trigger plates 660 and 670 towards each other results in axial rotation of the drive shaft 680 about the drive shaft axis 646A. Axial rotation of the drive shaft assembly 646 in turn results in axial rotation of the first transfer gear about the drive shaft axis 646A.

The fifth example surgical tool 620 may be used in a manner substantially the same as the first and second example surgical tools 20 and 320 as described above. The use of two sets of rack projections 664 and 674 determines the activation force on the tool portion provided the surgeon's fingers against one of the trigger members 642 and 644 and the opposing activation force by the surgeon's thumb against the other of the trigger members 642 and 644. These opposing activation forces will be balanced between the fingers or the thumb.

### VI. Sixth Example Surgical Tool

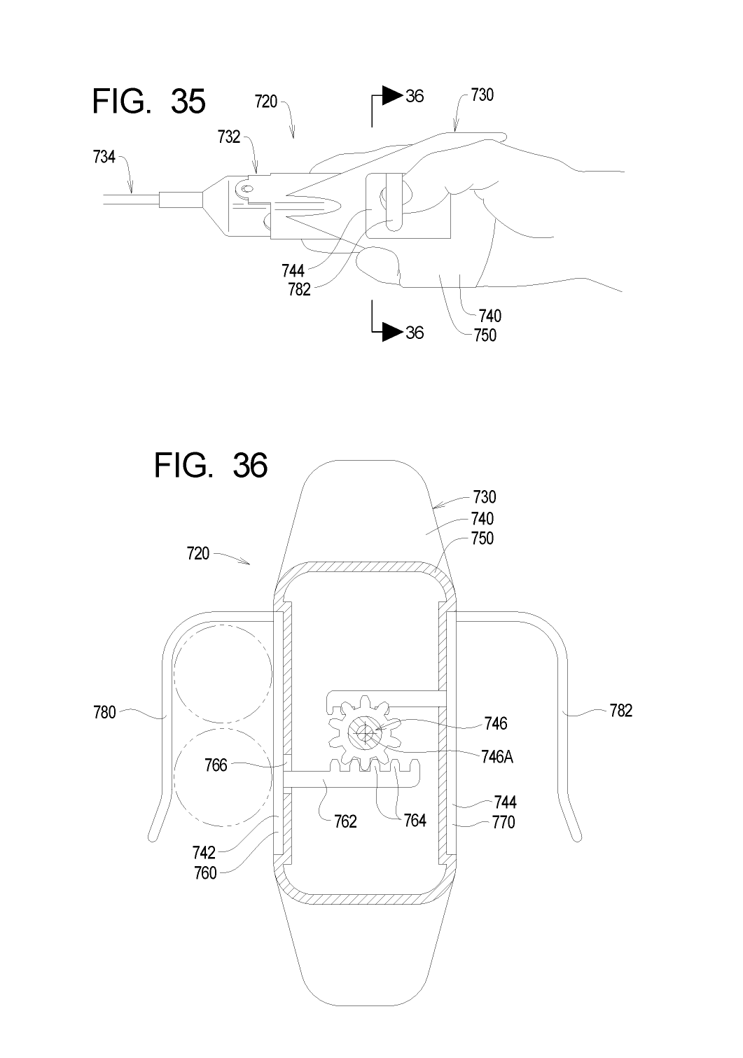

Referring now to FIGS. 35 and 36 of the drawing, depicted therein is a sixth example surgical tool 720 comprising a base portion 730 defining a base axis 730A, a transfer portion 732 defining a transfer axis, and a tool portion 734 defining a tool axis. With the base portion 730 grasped by the surgeon's hand, a neutral pinching motion of the user's fingers towards the user's thumb causes the fingers to move along the first secondary hand axis and the thumb to move along the second secondary hand axis.

The example base portion 730 comprises a base housing 740, a first trigger member 742, a second trigger member 744, and a drive shaft assembly 746 defining a drive shaft axis 746A. The base housing 740 comprises a base housing outer wall 750 that supports first and second pivot pins (not shown) to allow the first and second trigger members 742 and 744 to rotate about first and second trigger axes, respectively. The first trigger member 742 defines a first trigger plate 760 and a rack projection 762 defining a plurality of rack teeth 764. A rack opening 766 is formed in the base outer wall 750. The second trigger member 744 defines a second trigger plate 770. The first and second trigger plates 760 and 770 are pivotably supported to the base housing outer wall 750. So supported, the rack projection 764 extends through the rack opening 768 and interior of the base housing 740 such that the rack teeth 764 engage the drive shaft assembly 746.

In the sixth example surgical tool 720, first and second plate braces 780 and 782 extend from the first and second pivot plates 760 and 770, respectively. The plate braces 780 and 782 are sized and dimensioned to extend over and snugly receive outer portions of the surgeon's thumb and fingers while inner portions of the surgeon's thumb and fingers are against the first and second pivot plates 760 and 770. Movement of the surgeon's thumb and fingers in both directions is thus transmitted to first and second pivot plates 760 and 770. In the sixth example surgical tool, movement of the first pivot plate 760 in both directions causes movement of the rack projection 764 in two directions and thus operation of the drive shaft assembly 746.

The sixth example surgical tool 720 may be used in a manner substantially the same as the first and second example surgical tools 20, 320, 420, 520, and 620 as described above. In particular, plate braces such as the example plate braces 780 and 782 may be applied to the pivot plates of any of the example tools described herein, including the tools 20, 320, 420, 520, 620, 820, and 920, to facilitate movement of the pivot plates in both directions. Use of plate braces such as the example plate braces 780 and 782 may obviate the need for a trigger spring to bias the trigger plates against the force of the surgeon's thumb and fingers into a home position.

### VII. Seventh Example Surgical Tool

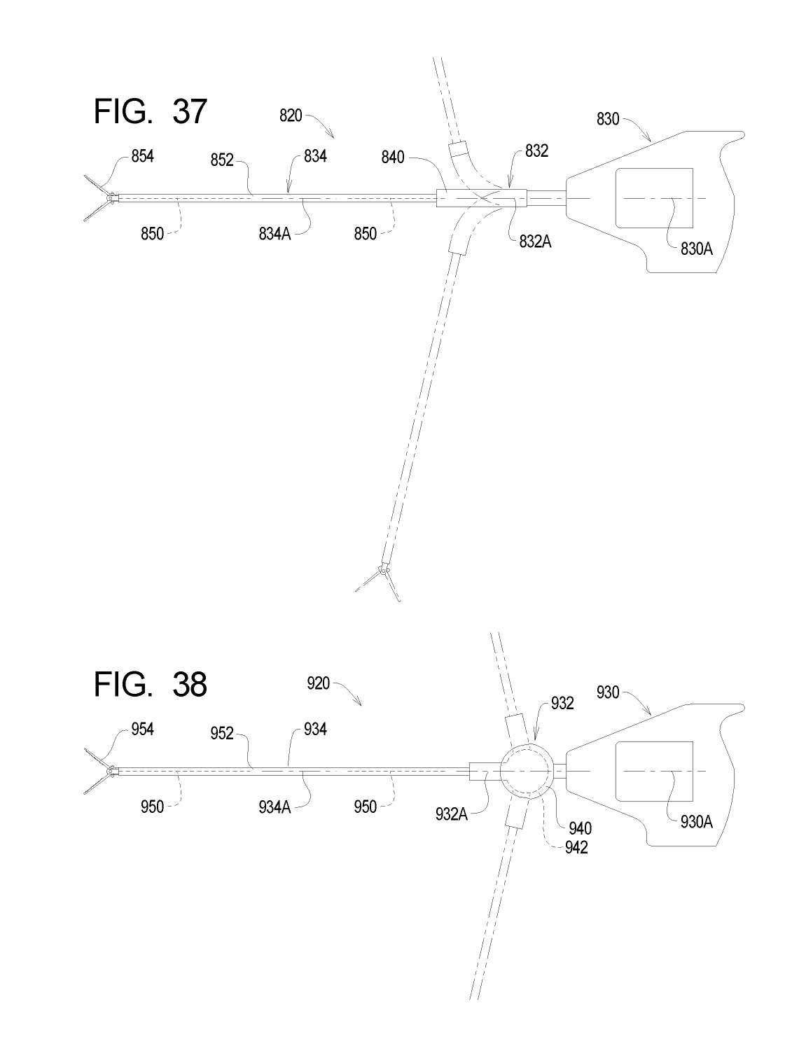

Referring now to FIG. 37 of the drawing, depicted therein is a seventh example surgical tool 820 comprising a base portion 830 defining a base axis 830A, a transfer portion 832 defining a transfer axis 832A, and a tool portion 834 defining a tool axis 834A. With the base portion 830 grasped by the surgeon's hand, a neutral pinching motion of the user's fingers towards the user's thumb causes the fingers to move along the first secondary hand axis and the thumb to move along the second secondary hand axis. The base portion 830 and tool portion 834 are or may be constructed in the same manner as the transfer portion and tool portion of the example surgical tools 20, 320, 420, 520, 620, and 720 described above and will not be described in detail herein.

And as with the example surgical tools 20, 320, 420, 520, 620, and 720 described above, the transfer axis 832A and thus the tool axis 834A may be placed at a pivot angle relative to the base axis 830A such that the surgeon may hold the seventh example surgical tool 820 in the neutral, comfortable position during most surgical situations. In particular, during use the tool axis 834A is arranged relative to the transfer axis 832A such that the angle at which the tool axis 834A extends relative to the base axis 830A may also be changed. Further, the tool portion 834 may be axially rotated about the tool axis 834A. The seventh example surgical tool 820 thus may be reconfigured as necessary to reduce stress on the surgeon's hand and thus the likelihood of repetitive use injuries to the surgeon.

FIG. 37 illustrates that the transfer portion 832 comprises a resilient connector member 840 that connects the base portion 830 to the tool portion 834. The resilient connector member 840 is deformable as shown in FIG. 37 to allow the tool axis 834A to be moved to any tool angle within approximately a 90° arc relative to the base axis 830A. The tool portion 834 comprises a tool cable 850, a hollow tool jacket 852, and a tool 854. The tool cable 850 extends between the base portion 830 and the tool 854 through the tool jacket 852. The tool cable 850 is capable of bending to accommodate deformation of the connector member 840 but is sufficiently rigid to transfer axial rotation created by the base portion 830 to the tool 854, resulting in proper operation of the tool 854.

The example transfer portion 832 may be adapted for use with any of the surgical tools 20, 320, 420, 520, 620, and 720 described above.

### VIII. Eighth Example Surgical Tool

Referring now to FIG. 38 of the drawing, depicted therein is an eighth example surgical tool 920 comprising a base portion 930 defining a base axis 930A, a transfer portion 932 defining a transfer axis 932A, and a tool portion 934 defining a tool axis 934A. With the base portion 930 grasped by the surgeon's hand, a neutral pinching motion of the user's fingers towards the user's thumb causes the fingers to move along the first secondary hand axis and the thumb to move along the second secondary hand axis. The base portion 930 and tool portion 934 are or may be constructed in the same manner as the transfer portion and tool portion of the example surgical tools 20, 320, 420, 520, 620, 720, and 820 described above and will not be described in detail herein.

And as with the example surgical tools 20, 320, 420, 520, 620, 720, and 820 described above, the transfer axis 932A and thus the tool axis 934A may be placed at a pivot angle relative to the base axis 930A such that the surgeon may hold the sixth example surgical tool 920 in the neutral, comfortable position during most surgical situations. In particular, during use the tool axis 934A is arranged relative to the transfer axis 932A such that the angle at which the tool axis 934A extends relative to the base axis 930A may also be changed. Further, the tool portion 934 may be axially rotated about the tool axis 934A. The eighth example surgical tool 920 thus may be reconfigured as necessary to reduce stress on the surgeon's hand and thus the likelihood of repetitive use injuries to the surgeon.

FIG. 38 illustrates that the transfer portion 932 comprises first and second discs 940 and 942 that connects the base portion 930 to the tool portion 934. The first and second discs 940 and 942 are rotatable relative to each other as shown in FIG. 38 to allow the tool axis 934A to be moved within and fixed at any tool angle within approximately a 90° arc relative to the base axis 930A. The discs 940 and 942 may be held at a desired angle by a lock system that employs friction, cam surfaces, opposing teeth, and/or cam springs. Alternatively, the discs 940 and 942 may be allowed to rotate freely relative to each other so that the tool angle may be changed as necessary during use of the tool 920.

The tool portion 934 comprises a tool cable 950, a hollow tool jacket 952, and a tool 954. The tool cable 950 extends between the base portion 930 and the tool 954 through the tool jacket 952. The tool cable 950 is capable of bending to accommodate movement of the discs 940 and 942 relative to each other but is sufficiently rigid to transfer axial rotation created by the base portion 930 to the tool 954, resulting in proper operation of the tool 954.

The example transfer portion 932 may be adapted for use with any of the surgical tools 20, 320, 420, 520, 620, 720, and 820 described above.

What is claimed is:

1. A surgical tool comprising:
a base portion defining a base axis and comprising at least one trigger member;
a tool portion defining a tool axis and comprising at least one tool assembly; and
a transfer portion operatively connected between the base portion and the tool portion such that
a pivot angle between the base axis and the tool axis may be altered; and
movement of the at least one trigger member causes movement of the tool assembly.

2. A surgical tool as recited in claim 1, in which the tool portion comprises a tool rod and a tool, where the tool rod operatively connects the at least one trigger member to the tool such that displacement of the at least one trigger member causes movement of the tool.

3. A surgical tool as recited in claim 1, in which the base portion comprises a drive shaft operatively coupled to the tool assembly, where displacement of the at least one trigger member causes axial rotation of the drive shaft, resulting in movement of the tool.

4. A surgical tool as recited in claim 2, in which the base portion comprises a drive shaft operatively coupled to the tool rod, where displacement of the at least one trigger member causes axial rotation of the drive shaft and axial rotation of the drive shaft causes axial rotation of the tool rod, resulting in movement of the tool.

5. A surgical tool as recited in claim 1, in which:
the base portion comprises first and second trigger members;
the first and second trigger members are supported for movement relative to each other; and
a surgeon's fingers engage and displace the first trigger member; and
a surgeon's thumb engages and displaces the second trigger member.

6. A surgical tool as recited in claim 5, in which:
movement of the surgeon's fingers define a first secondary axis of a surgeon's hand;
movement of the surgeon's thumb define a second secondary axis of the surgeon's hand; and
the first and second trigger members are supported for movement such that, when the surgical tool is held by the surgeon's hand during normal use,
the first trigger member moves along the first secondary axis, and
the second trigger member moves along the second secondary axis.

7. A surgical tool as recited in claim 1, in which:
the base portion comprises a drive assembly operatively coupled to the at least one tool assembly;
the at least one trigger member defines a projection; and
the projection engages the drive assembly such that movement of the at least one trigger member causes movement of the at least one tool assembly.

8. A surgical tool as recited in claim 7, in which:
the drive assembly comprises at least one drive gear;
the projection comprises a rack; and
the rack engages the at least one drive gear to cause movement of the at least one tool assembly.

9. A surgical tool as recited in claim 8, in which the drive gear is supported by a drive shaft operatively coupled to the at least one tool assembly, where the rack engages the drive gear to cause axial rotation of the drive shaft and thus movement of the at least one tool assembly.

10. A surgical tool as recited in claim 5, in which:
the base portion comprises a drive assembly operatively coupled to the at least one tool assembly;
the first trigger member defines a first projection;
the second trigger member defines a second projection; and
the first and second projections engages the drive assembly such that movement of the at least one trigger member causes movement of the at least one tool assembly.

11. A surgical tool as recited in claim 10, in which:
the drive assembly comprises at least one drive gear;
the first projection comprises a first rack;
the second projection comprises a second rack; and
the first and second racks engage the at least one drive gear to cause movement of the at least one tool assembly.

12. A surgical tool as recited in claim 10, in which:
the drive assembly comprises first and second drive gears;
the first projection comprises a first rack;
the second projection comprises a second rack; and
the first and second racks engage first and second drive gears, respectively, to cause movement of the at least one tool assembly.

13. A surgical tool as recited in claim 11, in which the at least one drive gear is supported by a drive shaft operatively coupled to the at least one tool assembly, where the first and second racks engage the at least one drive gear to cause axial rotation of the drive shaft and thus movement of the at least one tool assembly.

14. A surgical tool as recited in claim 12, in which the first and second drive gears are supported by a drive shaft operatively coupled to the at least one tool assembly, where the first and second racks engage the first and second drive gears, respectively, to cause axial rotation of the drive shaft and thus movement of the at least one tool assembly.

15. A surgical tool as recited in claim 1, in which:
the drive assembly comprises a drive shaft defining at least one drive gear and a first transfer gear;
the at least one trigger member comprises a projection defining a rack;
the at least one tool assembly comprises a second transfer gear;
the rack engages the at least one drive gear to cause rotation of the first transfer gear; and
the first transfer gear engages the second transfer gear to cause movement of the at least one tool assembly.

16. A surgical tool as recited in claim 15, in which:
the base portion defines a base axis;
the at least one tool assembly defines a tool axis;
the second transfer gear is supported by a tool rod;
the first and second transfer gears define first and second sets of conical teeth, respectively; and
when disengaged, the first and second transfer gears allow the pivot angle between the base axis and the tool axis to be changed; and
when engaged, the first and second transfer gears transfer rotation of the drive shaft to the tool rod through the pivot angle.

17. A surgical tool as recited in claim 16, a lock assembly for maintaining a desired pivot angle.

18. A surgical tool for use by a surgeon having a hand defining a first secondary axis defined by movement of a surgeon's fingers and a second secondary axis defined by movement of a surgeon's thumb, the surgical tool comprising;
a base portion defining a base axis and comprising first and second trigger members;
a tool portion defining a tool axis and comprising at least one tool assembly; and
a transfer portion operatively connected between the base portion and the tool portion such that movement of the at least one trigger member causes movement of the tool assembly, wherein
during use of the surgical tool,
the first trigger member is supported for movement along the first secondary axis, and
the second trigger member is supported for movement along the second secondary axis.

19. A method of allowing a surgeon having a hand defining a first secondary axis defined by movement of a surgeon's fingers and a second secondary axis defined by movement of a surgeon's thumb to perform surgery, the method comprising the steps of:
providing a base portion defining a base axis;
supporting first and second trigger members for movement relative to the base portion;
providing a tool portion defining a tool axis and comprising at least one tool assembly;
operatively connected the base portion and the tool portion such that movement of the first and second trigger members causes movement of the tool assembly;
holding the base portion in the surgeon's hand such that
the first trigger member is supported for movement along the first secondary axis, and
the second trigger member is supported for movement along the second secondary axis; and
displacing the first and second trigger members along the first and second secondary axes to operate the at least one tool assembly.
